(19) United States
(12) Patent Application Publication
(10) Pub. No.: US 2013/0085468 A1
(43) Pub. Date: Apr. 4, 2013

(54) CATHETER WITH BODY WALL SEPARATOR

(71) Applicant: YURI BUYDENOK, Moscow (RU)

(72) Inventor: YURI BUYDENOK, Moscow (RU)

(21) Appl. No.: 13/398,433

(22) Filed: Feb. 16, 2012

Related U.S. Application Data
(60) Provisional application No. 61/542,654, filed on Oct. 3, 2011.

(51) Int. Cl. A61M 25/00; A61M 27/00

(52) U.S. Cl. 604/500; 604/540; 604/264

(57) ABSTRACT

A catheter comprising a body wall separator for preventing the occlusion of catheter openings by body tissues when the catheter is placed in the body of a subject. The inventive catheter provides enhanced flow of fluids to and from the body. Methods of making and using the catheter, for drainage purposes for example, are also within the scope of the invention.

This application claims priority to provisional application Ser. No. 61/542,654 filed Oct. 3, 2011, the entire contents of which are incorporated herein by reference.

### BACKGROUND

The invention generally relates to catheters for transferring fluids into or out of the body. More particularly, the invention relates to a catheter comprising a body wall separator for improving the transfer of fluids into or out of the body.

Catheters known in the art have a distinct disadvantage in that tissues can obstruct or block the openings in the catheter when the catheter is placed in the body. Some devices have been introduced which address this problem. For example, Blake® surgical drains are white, radiopaque silicone drains comprising four open channels that are co-linear with a solid core center. However, Blake® surgical drains cannot be used with suction since the open channels prevent the formation of a vacuum in the drain's lumen. In addition, the open channels in the Blake® drain have an edge which can injure and pinch tissues in the body.

Pigtail catheters are another known form of catheter (e.g. drain). Pigtail catheters generally comprise a hook at their terminal end. The pigtail design presents a blunt, smooth, resilient end which reduces the risk of damage to tissues that come into contact with the catheter. While the curved design of the pigtail prevents tissue trauma, it fails to address the problem of body tissues obstructing the openings of the catheter and the communication of fluids into the lumen, This is particularly problematic in drainage catheters which rely on vacuum, body pressure or gravity to promote the flow of bodily fluids into the catheter and out of the body.

Others have approached the problem of poor catheter drainage and tissue obstruction by increasing the size of the catheter that is used in the body. Larger catheters however require larger surgical openings and increase tissue trauma in the body.

What is needed in the art therefore is a catheter that can safely occupy a body space without having body tissues obstruct the flow of fluids into and/or out of the catheter.

### SUMMARY OF THE INVENTION

The invention overcomes problems with known catheters by providing a frame, or body wall separator, for keeping body tissues away from the catheter's openings. Catheters of the invention improve the transfer of fluids to and from the body despite compressive forces by tissues which would otherwise block the openings of catheters known in the art.

It is therefore an object of the invention to safely improve the transfer of fluids into and out of the body by providing a catheter comprising a tubular body having at least one perforation, and a coil connected to said tubular body, wherein said coil wraps around said tubular body in a manner that prevents the at least one perforation from being obstructed by body tissues when the catheter is placed in the body of a subject. Said coil allows the distal end of the catheter of the invention (and said at least one perforation) to be threaded into a body space. Once the entire portion of the coil is threaded into the body space, the coil resumes its natural shape and winds around the tubular body, and the at least one perforation, thereby forming a barrier that protects the at least one perforation from obstruction by body tissues. When the catheter of the invention is withdrawn from the body, the flexibility in the coil allows it to unwind and assume a linear shape thereby decreasing the profile (e.g. width) of the catheter and preventing tissue trauma from its removal.

It is also the object of the invention to improve the transfer of fluids into and out of the body by providing a method comprising contacting a body space with a catheter of the invention, and transferring fluids into and/or out of the body space through said catheter.

Further objects of the invention include methods of making a catheter comprising, for example, providing a tubular body having a shape defined by a body wall, wherein said tubular body has at least one hole in said body wall near an end of said tubular body, and connecting a flexible coil to said end of said tubular body, wherein said coil is configured to wrap around said tubular body and said at least one hole thereby creating a barrier for protecting said at least one hole from being obstructed by tissues when said end of said tubular body is placed into the body of a subject.

### BRIEF DESCRIPTION OF THE DRAWINGS

FIG. 1 is planar view of a catheter according to the invention.

FIG. 2 is planar view of a catheter according to the invention wherein a body wall separator is in a stretched configuration.

FIG. 3 is planar view of a catheter according to the invention wherein a body wall separator is stretched over the shaft of said catheter.

FIGS. 4A-D are planar views of a catheter according to the invention in which a threading function of the catheter is depicted.

FIGS. 5A-D are planar views of a catheter according to the invention in which a retracting function of the catheter is depicted.

FIG. 6 is a perspective view of a catheter according to the invention as it occupies a body space in a human subject.

FIG. 6A is an exploded, partial view of the catheter from FIG. 6.

FIGS. 7-9 are planar views of various embodiments of the catheter of the invention,

FIG. 10 is a planar view of a catheter according to the invention, wherein a space between the coils in the body wall separator is depicted.

FIG. 11 is an end-view of the distal end of a catheter according to the invention, wherein a space between the coils of the body wall separator and the catheter shaft is depicted.

FIG. 12 is a functional view of a catheter according to the invention as it occupies a body space in a human subject, wherein fluids are depicted flowing from the body of said subject.

FIG. 13 is a functional view of a catheter according to the invention, wherein the catheter is being withdrawn from the body of a human subject.

FIGS. 14 and 15 are close-up views of catheters according to the invention, wherein the catheters are in contact with body tissues.

### DETAILED DESCRIPTION

The accompanying figures are provided to describe preferred embodiments of the invention and are not intended to limit the same.

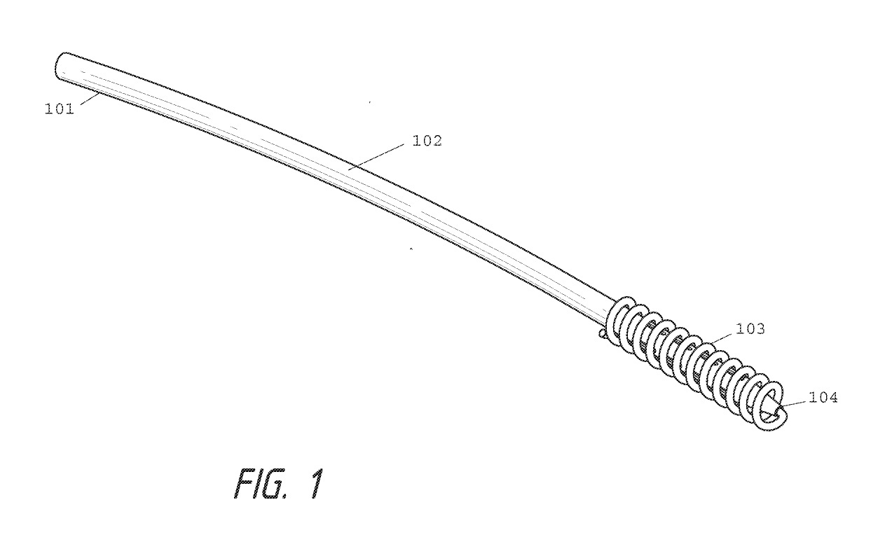

FIG. 1 shows an embodiment of a catheter according to the invention comprising shaft (102) having proximal end (101) and distal end (104). In the embodiment shown in FIG. 1, body wall separator (103) is connected to distal end (104) and wrapped (i.e. coiled) around shaft (102). One skilled in the art will appreciate that body wall separator (103) may be connected to distal end (104) by any suitable connection means including, but not limited to, threads, interlocking components (e.g. a locking mechanism), welding, adhesive, or combinations thereof. It is further contemplated that body wall separator (103) and distal end (104) may comprise a single, continuous material, such as that produced by molding the coil and shaft as a single component, for example. Shaft (102) is a hollow member defined by a shaft wall surrounding a lumen that permits fluids to flow freely through shaft (102) and exit out an outflow opening (not shown) located at proximal end (101). The lumen of shaft (102) may also comprise capillaries for conducting fluid via capillary action. Shaft (102) may assume the configuration of a tube with an annular opening at proximal end (101), for example. Proximal end (101) may assume an annular opening, or any configuration that permits proximal end (101) to be connected to a separate outflow tube, a suction or positive pressure device (e.g. pump or syringe), a drainage vessel, or hydrophilic mesh, for example. It is of course contemplated that shaft (102) may assume varying lengths depending upon, for example, the proximity and type of equipment to be used in transferring fluids into and out of the body and/or the depth of the body space that the catheter is intended to occupy.

Catheters of the invention are preferably manufactured from a material, or combination of materials, that provides the catheter with sufficient flexibility to be shaped and manipulated in a manner as disclosed herein. Suitable materials for manufacturing the catheter of the invention include, but are not limited to, rubber (e.g. silicone rubber and latex), urethane, neoprene, vinyls, or combinations thereof. It is contemplated that body wall separation (103) and shaft (102) may have the same or different levels of flexibility. For example, shaft (102) may have less flexibility than body wall separator (103) to facilitate the wrapping of body wall separator (103) around shaft (102), and to allow the catheter to be threaded into and/or pushed into a body space. It is therefore contemplated that distal end (104) may be pushed into a body space, instead of or in addition to, threading distal end (104) into the body of a subject.

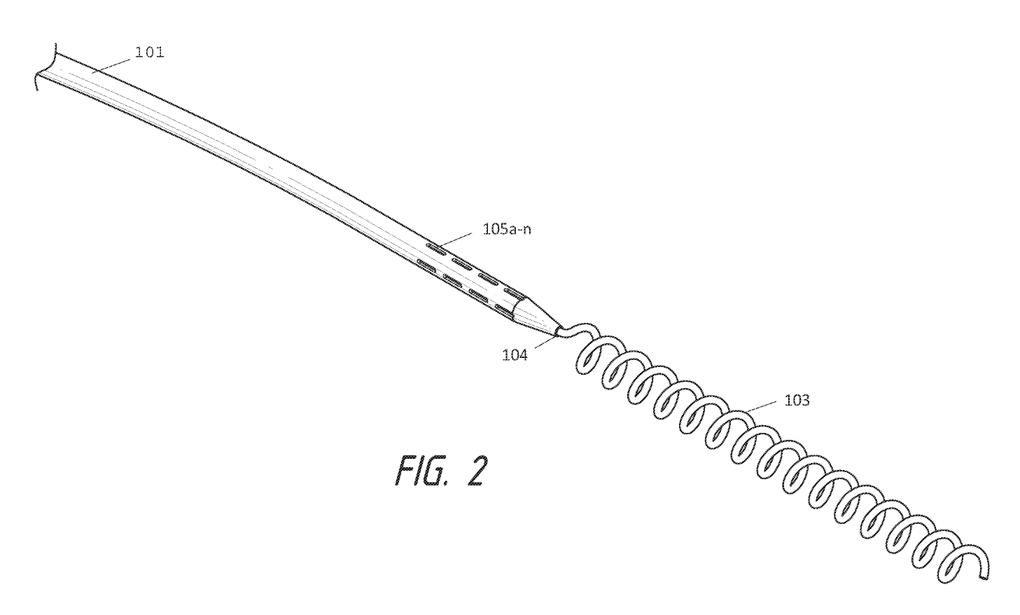

FIG. 2 depicts an embodiment of a catheter of the invention wherein body wall separator (103) is in a stretched, open configuration. Body wall separator (103) may assume this deformed configuration when force is applied to stretch body wall separator (103) from its coiled, natural state. Being made of a flexible material, body wall separator (103) assumes a permanent coiled shape when not being forced into a different configuration. Body wail separator (103) preferably comprises a flexible material that is manufactured to have the shape memory of a coil. Body wall separator (103) may comprise, for example a solid (or tubular) linear member having the shape memory of a coil.

Also depicted in FIG. 2 are a series of openings, or perforations (105a-n) for placing the lumen of shaft (102) in fluid communication with a body space as the catheter's distal end (104) occupies the body of a subject. That is, openings (105a-n) permit fluids to flow through the lumen of catheter shaft (102) into and/or from a body space in a subject. Openings (105a-n) may assume any shape, configuration, distribution or number that permits a desired fluid or substance to flow into, and/or out of, the lumen of shaft (102). As used herein, the term “fluid” includes, but is not limited to, interstitial fluids, washing fluid, asceptic fluids, antibiotics, and/or chemotherapy solutions. Fluids may contain substances (e.g. particles) such as, for example, clots, particles (e.g. post-surgical wash), necrotic tissue, post-endscopic surgery parts, etc. Openings (105a-n) may assume shapes selected from for example, round, elliptical, square, and combinations thereof.

One skilled in the art will appreciate that the number and size of openings (105a-n) may be selected to achieve a desired level of flow into, and/or out of, the lumen of the catheter. In addition, the size of the openings may be selected to allow the passage of particles suspended in a fluid. In some non-limiting, exemplary embodiments of the invention, openings (105a-n) are between about 0.1 mm to 10 cm, Openings (105a-n) are preferably positioned near the distal end of shaft (102) so as to permit openings (105a-n) to occupy a body space when distal end (104) is positioned within the body of a subject as disclosed herein. Openings (105a-n) may occupy a length of shaft (102) that is the same as, less than, or more than, the length of body wall separator (103) (when body wall separator (103) is in its natural state). In an aspect of the invention, openings (105a-n) may be arranged symmetrically (or asymmetrically) on one side of shaft (102), or around shaft (102).

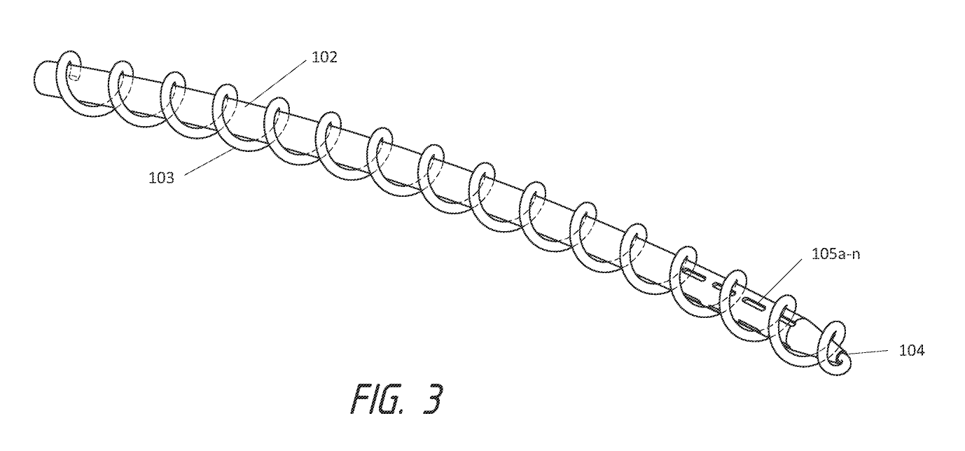

FIG. 3 shows an embodiment of the catheter of the invention wherein body wall separator (103) is wrapped around shaft (102) and stretched towards the catheter's proximal end (101). In this configuration, the catheter is in a shape that permits it to be threaded into a surgical opening in the body of a subject, for example. In this stretched, wrapped configuration, body wall separator (103) functions as a set of threads that draws shaft (102) into a body space when turned in a screwing motion. Body wall separator (103) may comprise any number of helical turns provided that such number of turns permits the catheter of the invention to be used as disclosed herein.

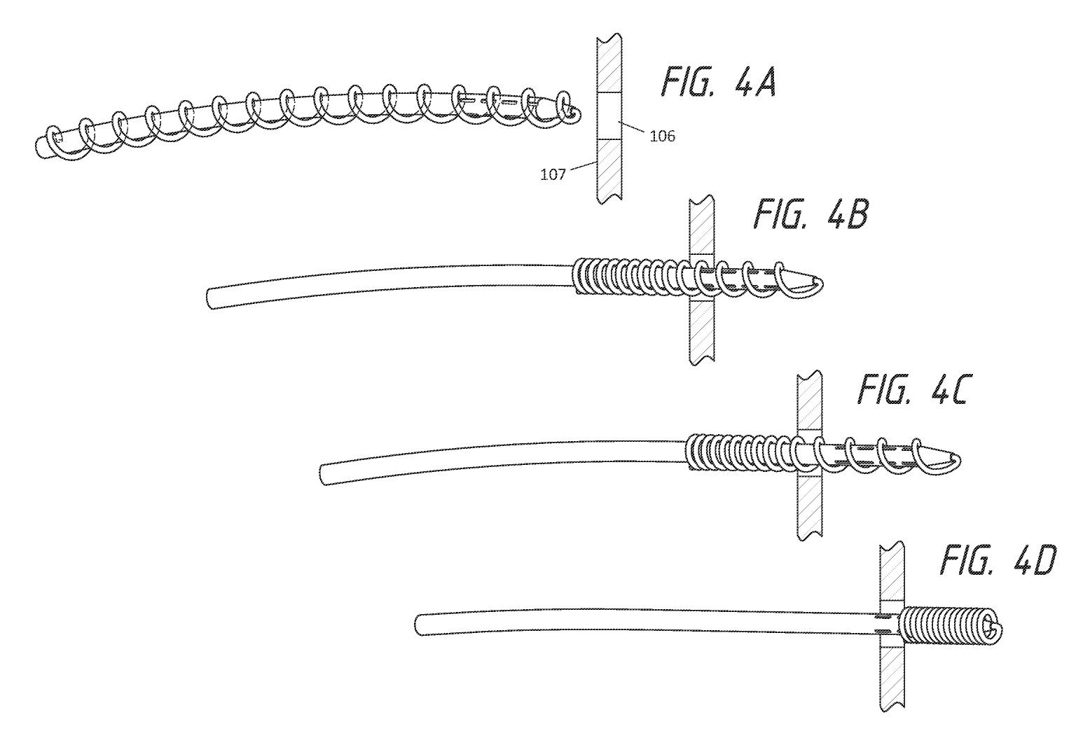

FIGS. 4A-D show how a catheter of the invention may be threaded into the body of a subject. Depicted are a body wall (107) and surgical opening (106). Body wall (107) comprises the surface of a body space such as the skin or stomach wall, for example, while surgical opening (106) may comprise, for example, an incision that may be created for placement of a catheter of the invention into a body, or as the result of a surgical procedure (e.g. endoscopic surgical opening). FIG. 4A shows a catheter of the invention with body wall separator (103) in a stretched configuration as it approaches surgical opening (106). FIGS. 4B-C show the advancement of the catheter into a body space through surgical opening (106). As shown in FIG. 4D, body wall separator (103) assumes its natural state once it is completely threaded through body wall (107). In its natural state, body wall separator (103) prevents tissues within the body from contacting openings (105a-n) thereby permitting the free passage of fluids through shaft (102).

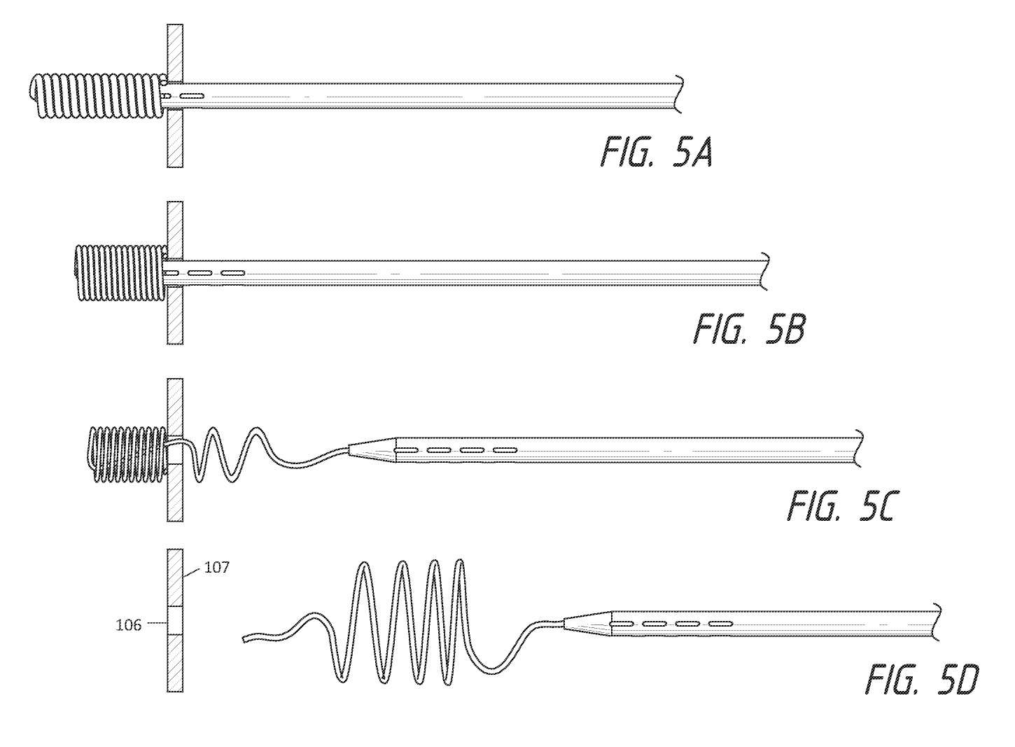

FIGS. 5A-D show how a catheter of the invention may be withdrawn from the body of a subject, FIG. 5A shows a catheter of the invention with body wall separator (103) in its natural state. FIG. 5B shows body wall separator (103) compressing as it is drawn out of the body of a subject. As depicted in FIGS. 5C-D, shaft (102) and the coils of body separator (103) pass through body wall separator (103), in succession, as the catheter is withdrawn from the body.

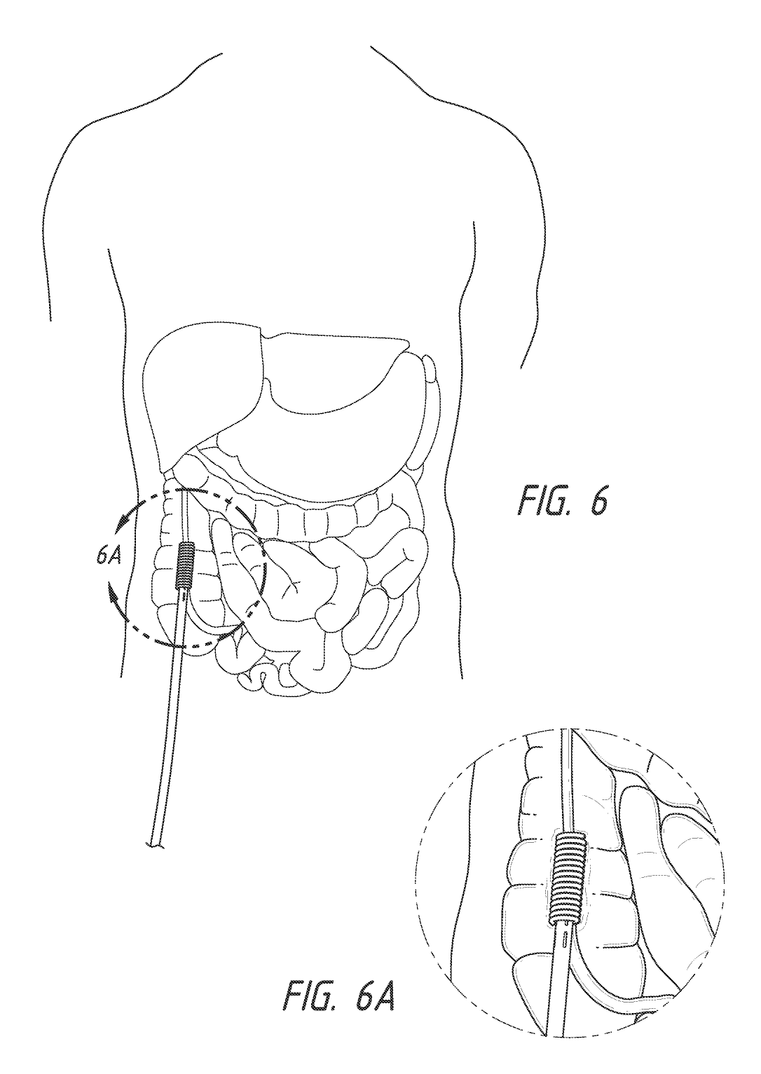

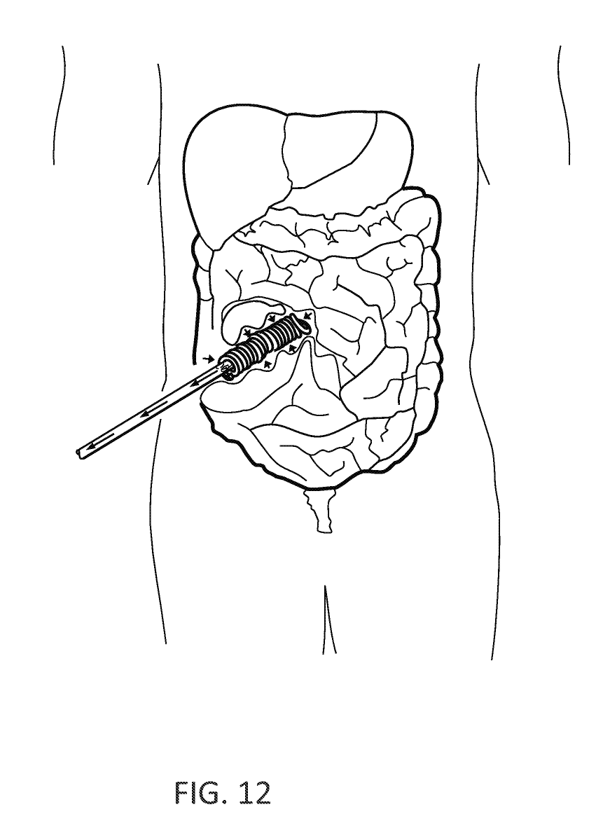

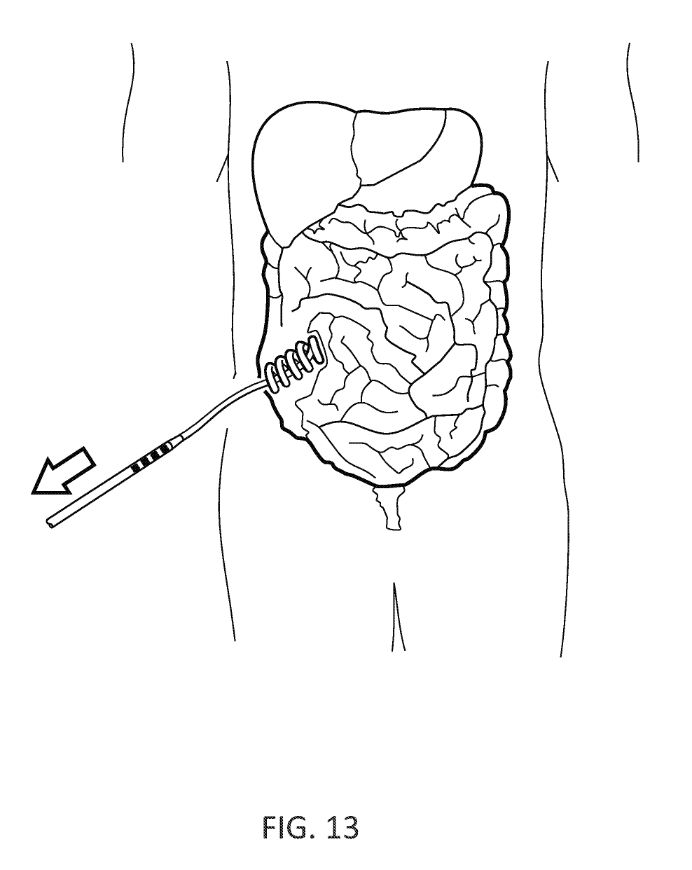

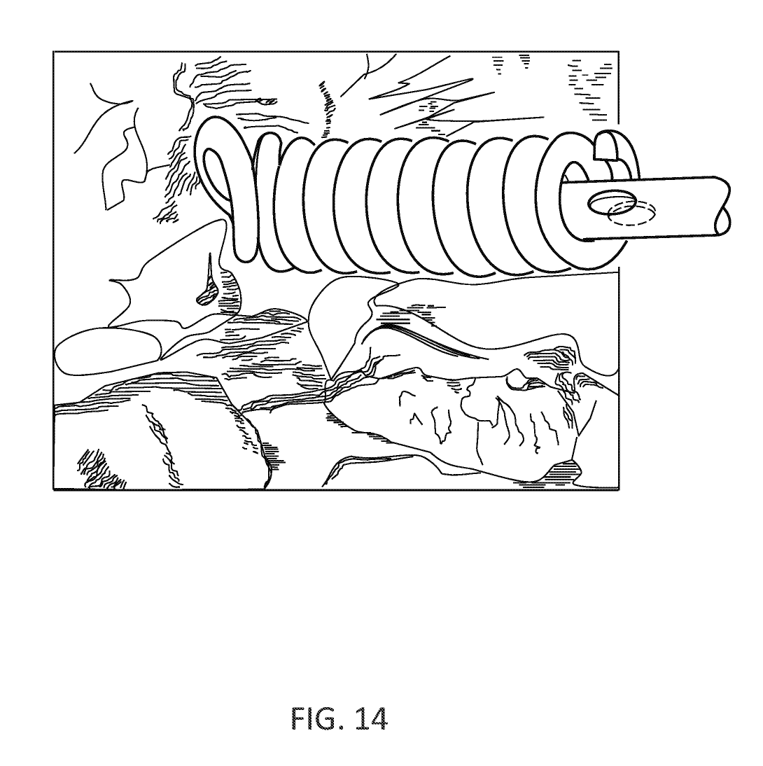

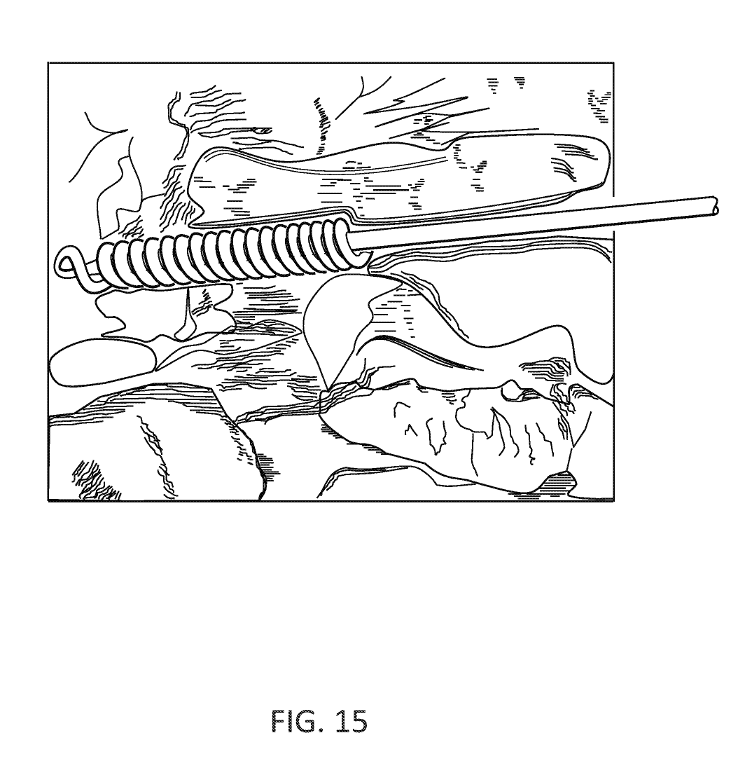

FIGS. 6 and 6A show the placement of the catheter within a body space of a human subject. As depicted, body wall separator (103) assumes its natural state white wrapped around shaft (102). In this configuration, body wall separator (103) prevents tissues from obstructing openings (105a-n) thereby permitting the free flow of fluids into shaft (102). FIG. 12 is another depiction of the catheter of the invention as it occupies a body space in a human subject. FIG. 12 depicts the catheter of the invention functioning as a surgical drain wherein body fluids pass through body wall separator (103), into openings (105a-n) and out shaft (102) for collection. FIG. 13 depicts an embodiment of the catheter of the invention wherein the catheter is being withdrawn from a human subject, As the catheter is withdrawn, body wall separator (103) uncoils to assume a linear configuration during its removal from the body. FIGS. 14 and 15 show embodiments of the catheter of the invention in situ. That is, FIGS. 14 and 15 depict how body wall separator (103) prevents the blocking of openings (105a-n) as body wall separator (103) is contacted (e.g. compressed) by tissues within a body space.

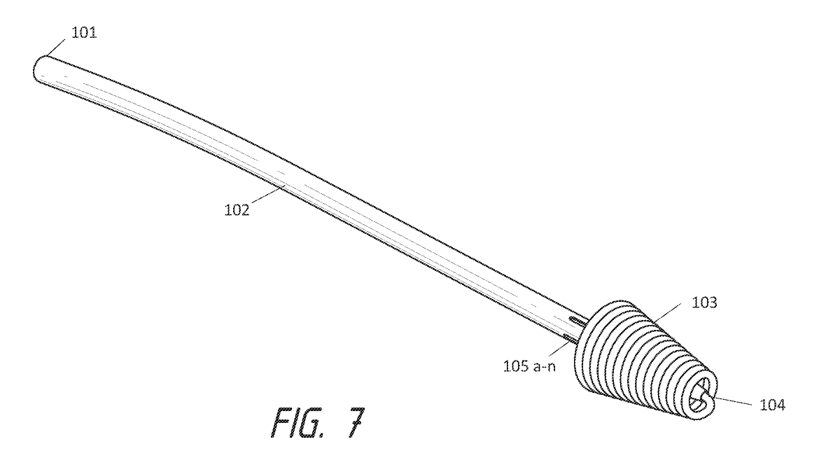

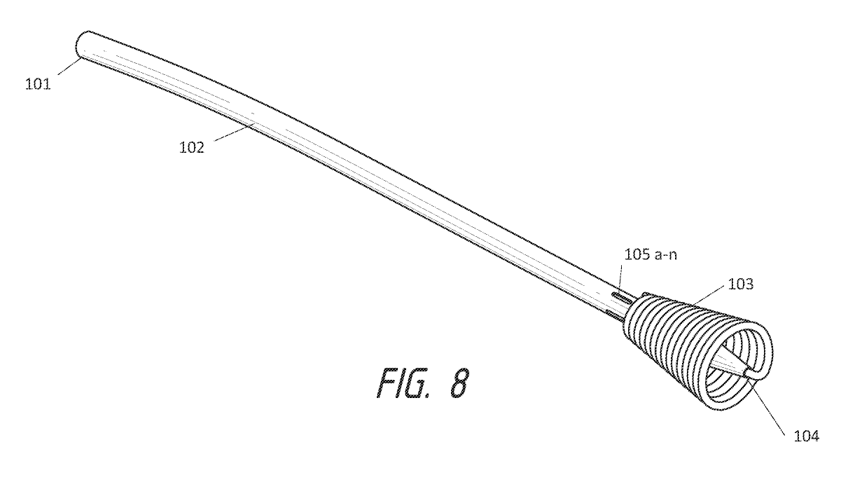

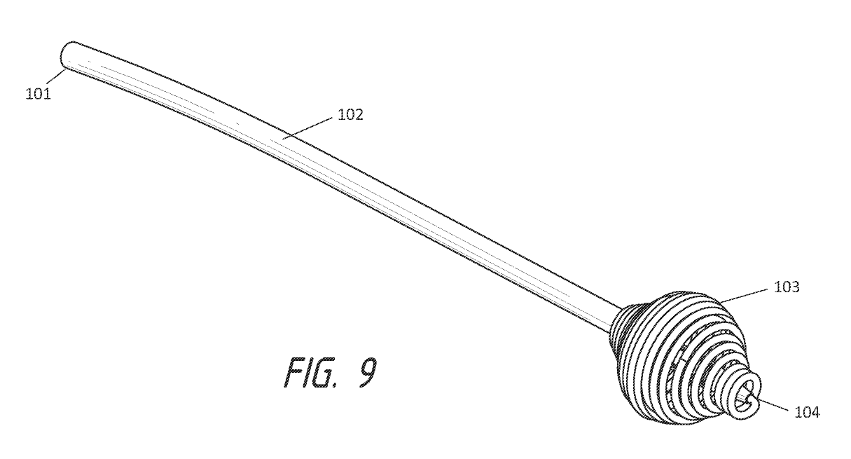

One aspect of the invention concerns the shape that body wall separator (102) assumes in its natural state. Body wall separator (103) may assume any shape that permits body wall separator (103) and a portion of shaft (102) (e.g. distal end (104)) to occupy a target (i.e. desired) body space in a subject. FIGS. 7-9 depict some non-limiting configurations for body wall separator (103). In some aspects of the invention, body wall separator assumes the shape of a body cavity that is intended to be occupied by the catheter. For example, body wall separator may assume a shape configured to occupy a body space intrasinularly, retrobulbarly, subchoroidally, intrathecally, intranasally, intra-rectally, submucosally, subcutaneously, intravenously, intravaginally or intrabdominally.

While the catheter of the invention is depicted as having a particular shape and design, one skilled in the art will appreciate that the catheter of the invention may assume any shape and design that permits it to prevent the occlusion of openings (105a-n) white the catheter occupies a body space as disclosed herein. For example, distal end (104) may assume a conical shape where it connects to body wall separator (103). Alternatively, distal end (104) may assume a blunt shape having a connecting means for attachment of body wall separator (103). Body wall separator may connect to terminal end (104), or to the side of shaft (102). Distal end (104) may be closed, such as by connection to body wall separator (103). It is also contemplated that terminal end may be open, wherein body wall separator (103) connects to the side of shaft (102), for example.

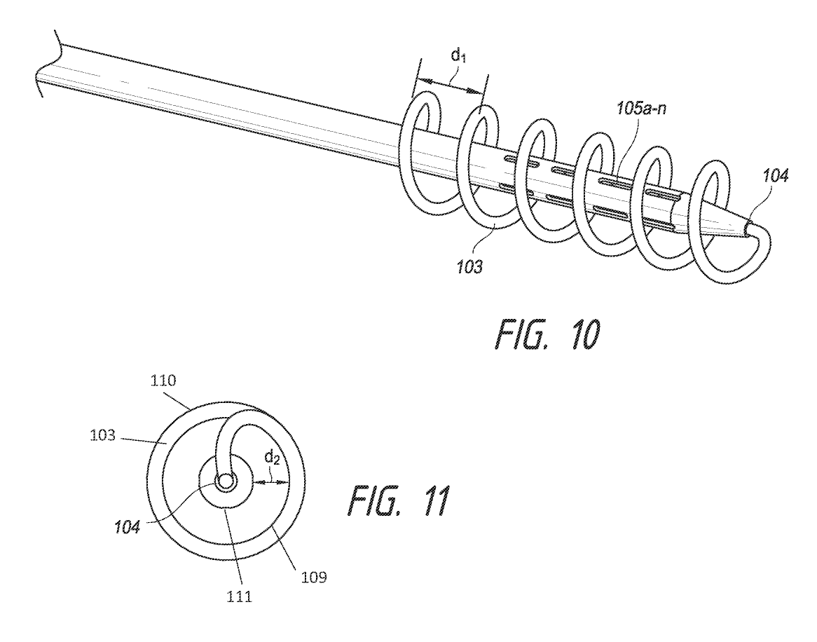

Another aspect of the invention concerns the dimension of the components of the inventive catheter. As shown in FIG. 10, the individual coils of body wall separator (103) may be separated by distance (d1) when body wall separator (103) is in its relaxed, natural state. One skilled in the art will appreciate that distance (d1) may vary according to the application for which the catheter is intended. For example, the individual coils (i.e.) helical turns may have no space between them such that they contact one another. Alternatively, the individual coils of body wall separator (103) may be spaced apart wherein distance (d1) ranges up to about 3 cm.

Another aspect of the invention concerns the distance of the outside of shaft (102) from the inside of body wall separator (103). FIG. 11 depicts a view from distal end (104) wherein body wall separator (103) comprises inner wall (109) and outer wall (110). The inside space of body wall separator (103) is therefore defined by inner wall (109). Shaft (102) similarly comprises shaft outer wall (111). In aspects of the invention, inner wall (109) is separated from shaft outer wall (111) by distance (d2) (when body wall separator (103) is in its natural state). Distance (d2) may assume any size that prevents the occlusion of openings (105a-n) when the catheter is placed in the body of a subject. It is also contemplated that inner wall (109) may be in contact with shaft outer watt (111) when body wall separator (103) is in its natural state. Distance (d2) may range up to about 2 cm, for example. All references to distances (d1) and (d2) herein assume that body wall separator (103) is in its natural, permanent shape.

In some practices, the catheter of the invention may assume a shape such as that depicted in FIG. 3 wherein body wall separator (103) is drawn towards proximal end (101) of shaft (102). Distal end (104) is then inserted into an opening in the body and threaded into the body space where the introduction and/or removal of fluids is desired. The catheter is threaded into the body until body wail separator (103) and openings (105a-n) are completely within the desired body space. Having entered the desired body space, body wall separator (103) resumes its natural shape such that it wraps around shaft (102) and openings (105a-n) such that openings (105a-n) are prevented from contacting tissues within the body space. Thus positioned, body wall separator (103), distal end (104) and at least a portion of shaft (102) occupy the desired body space. The remainder of shaft (102) remains outside the body for fluid communication with an implement such as a vacuum pump or collection vessel, for example.

The catheter of the invention may find use in applications where the removal of body fluids from a body space is desired. For example, the catheter of the invention finds use as a surgical drain for removing fluids from body compartments (e.g. body spaces) such as the abdominal cavity, or the spaces for which body wall separator (103) may be configured to occupy as discussed above. Some non-limiting uses of the catheter of the invention include irrigation systems such as the introduction of dialysis liquid into the abdominal cavity (and subsequent drainage of the same), interventional roentgen procedures of renal pelvis drainage (in ease of impaired urine outflow) and the drainage of pyogenic abscesses.

Thus having described preferred embodiments of the invention, alterations and modifications that do not depart from the spirit of the invention may occur to others. The invention is thus not limited to the preferred embodiments but is instead set forth in the appended claims and legal equivalents thereof.

We claim:

1. A catheter comprising:
a) a tubular body;
b) a coil connected to said tubular body; and
c) at least one .perforation in said tubular body.

2. The catheter of claim 1, wherein said coil is connected to an end of said tubular body, to the side of said tubular body, or a combination thereof.

3. The catheter of claim 1, wherein said coil comprises at least one helical turn.

4. The catheter of claim 1, wherein said at least one perforation located near an end of said tubular body.

5. The catheter of claim 1, wherein said at least one perforation comprises perforations of the same size and shape, or perforations of different sizes and shapes.

6. The catheter of claim 1, wherein said tubular body and said coil are separate components and said coil is connectable to said tubular body.

7. The catheter of claim 6, wherein said coil is connectable to said tubular body by threads, a locking mechanism, adhesive, welding or a combination thereof.

8. The catheter of claim 1, wherein said tubular body and said coil comprise a single, continuous material.

9. The catheter of claim 1, wherein said at least one perforation occupies a length of said tubular body that is the same as the length of said coil, less than the length of said coil, or more than the length of said coil.

10. The catheter of claim 1, wherein the circumference of the inside of said coil is at least half of the circumference of said tubular body.

11. The catheter of claim 1, wherein said coil comprises a flexible linear member having the memory of said coil.

12. The catheter of claim 1, wherein said coil surrounds said tubular body and said at least one perforation.

13. The catheter of claim 1, wherein a space separates the outside of said tubular body from said coil.

14. The catheter of claim 12, wherein said coil prevents said at least one perforation from contacting body tissues when said coil and at least a portion of said tubular body are positioned within the body of a subject.

15. A catheter for transferring fluids to or from the body of a subject, said catheter comprising:
a) a tubular member comprising a lumen; and
b) a coiled body connected to said tubular member;
c) wherein said tubular member comprises at least one perforation for placing said lumen in fluid communication with a space surrounding said tubular member.

16. The catheter of claim 15, wherein said at least one perforation is located near an end of said tubular member.

17. The catheter of claim 15, wherein said at least one perforation comprises perforations of the same size and shape, or perforations of different sizes and shapes.

18. The catheter of claim 15, wherein said tubular member and said coiled body are separate components and said coiled body is connectable to said tubular body.

19. The catheter of claim 18, wherein said tubular body is connectable to said coiled body by threads, a locking mechanism, adhesive, welding or a combination thereof.

20. The catheter of claim 15, wherein said tubular member and said coiled body comprise a single, continuous material.

21. The catheter of claim 15, wherein said at least one perforation occupies a length of said tubular member that is the same as the length of said coiled body, less than the length of said coiled body, or more than the length of said coiled body.

22. The catheter of claim 15, wherein said coiled body comprises a flexible linear member that forms said coiled body in its natural state.

23. The catheter of claim 15, wherein said coiled body comprises a plurality of helical turns and wherein said helical turns (a) contact one another, (h) are separated by a space, or a combination thereof.

24. The catheter of claim 15, wherein said coiled body surrounds said at least one perforation.

25. The catheter of claim 24, wherein a space separates said tubular member from said coiled body.

26. The catheter of claim 15, wherein said coiled body prevents said at least one perforation from contacting body tissues when said coiled body and at least a portion of said tubular member are positioned within the body of a subject.

27. A catheter comprising:
a) a tube having an inner wall and an outer wall, said inner wall defining a lumen inside said tube;
b) a flexible linear member connectable to an end of said tube, said linear member having the elastic memory of a coil having at least one helical turn; and
c) at least one perforation in said tube for placing said lumen in fluid communication with a space surrounding said tube, wherein said at least one perforation is located near an end of said tube.

28. The catheter of claim 27, wherein said linear member forms said coil and said coil surrounds said at least one perforation in said tube.

29. The catheter of claim 28, wherein a space separates said outer wall of said tube from said coil.

30. The catheter of claim 27, wherein said coil prevents body tissues from contacting said at least one perforation in said tube when said coil and at least a portion of said tube are placed in the body of a subject.

31. A method for transferring fluids in the body of a subject, said method comprising:
a) providing a catheter according to any one of claims 1-30;
b) contacting said catheter with a body space in said subject; and
c) permitting fluids to flow into said subject, and/or from said subject, through said catheter.
